(19) United States
(12) Patent Application Publication
(10) Pub. No.: US 2014/0087456 A1
(43) Pub. Date: Mar. 27, 2014

(54) Isolating Target Cells From A Biological Fluid

(71) Applicant: National University Of Singapore, Singapore (SG)

(72) Inventors: Chwee Teck Lim, Singapore (SG); Swee Jin Tan, Singapore (SG); Ali Asgar S. Bhagat, Singapore (SG)

(73) Assignee: National University Of Singapore, Singapore (SG)

(21) Appl. No.: 14/030,343

(22) Filed: Sep. 18, 2013

Related U.S. Application Data
(63) Continuation-in-part of application No. PCT/SG2012/000084, filed on Mar. 13, 2012.
Application No. PCT/SG2012/000084 is a continuation-in-part of application No. 12/765,576, filed on Apr. 22, 2010.
(60) Provisional application No. 61/172,250, filed on Apr. 24, 2009.

(30) Foreign Application Priority Data
Mar. 18, 2011 (SG) 201101977-5

(51) Int. Cl. G01N 33/49

(52) U.S. Cl. CPC G01N 33/491
USPC 435/309.1

(57) ABSTRACT

A micro-fluidic device operable to isolate target cells from a biological fluid comprises: an inlet operable to receive the biological fluid, the biological fluid comprising target cells and other components; a waste outlet operable to receive at least the other components of the biological fluid; a plurality of parallel arrays of cell isolation wells coupling the inlet with the waste outlet, each parallel array of cell isolation wells supporting a flow of the biological fluid from the inlet to the waste outlet in response to a pressure differential thereacross, each array of cell isolation wells comprising a plurality isolation wells, each isolation well being dimensioned to mechanically trap the target cells therein whilst permitting flow of other components of the biological fluid; and at least one pressure maintenance structure operable to assist in maintaining a predetermined pressure differential across each of the plurality of parallel arrays of cell isolation wells.

### RELATED APPLICATIONS

This application is a continuation-in-part of International Application No. PCT/SG2012/000084, which designated the United States and was filed on Mar. 13, 2012, published in English, which claims the benefit of Singapore Application No. 201101977-5, filed Mar. 18, 2011. This application is also a continuation-in-part of U.S. application Ser. No. 12/765,576, filed Apr. 22, 2010, which claims the benefit of U.S. Provisional Application No. 61/172,250, filed on Apr. 24, 2009. The entire teachings of the above applications are incorporated herein by reference.

### FIELD

The present invention relates to a micro-fluidic device operable to isolate target cells from a biological fluid and a method of isolating target cells from a biological fluid.

### BACKGROUND

Cancer is a leading cause of death globally and early detection is one of the most effective means to combat the disease. Recent clinical studies show that the number of cancer cells in cancer patients' blood can predict the disease development and treatment efficacy. Studying these cells may also lead to a better understanding of the disease. Moreover, getting access to blood samples is relatively easy and less invasive and painful than tumour biopsies.

Isolation and enumeration of circulating tumour cells (CTCs) in peripheral blood has clinical significance in combating cancer (J. M. Reuben, S. Krishnamurthy, W. Woodward, M. Cristofanilli, Expert Opin. Med. Diagnostics 2, 339 (2008); S. Urtishak, R. K. Alpaugh, L. M. Weiner, R. F. Swaby, Biomarkers Med. 2, 137 (2008)). Deaths resulting from cancer are mainly due to late diagnosis of the disease and when metastasis has occurred (G. P. Gupta, J. Massague, Cell 127, 679-695 (2006); P. S. Steeg. Nat. Med. 12, 895 (2006)). To ensure patients receive timely treatment, enumerating CTCs in blood can complement existing early detection methods. Furthermore, blood samples being a routinely extracted body fluid in any health test can be easily attained to check for CTCs. CTCs are found in patients with metastatic carcinomas (W. J. Allard, J. Matera, M. C. Miller, M. Repollet, M. C. Connelly, C. Rao, A. G. Tibbe, J. W. Uhr, L. W. Terstappen, Clin. Cancer Res. 10, 6897 (2004); S. Steen, J. Nemunaitis, T. Fisher, J. Kuhn. Proc 21, 127 (2008), Bayl Univ Med Cent.) and are associated with the disease progression (M. Cristofanilli, G. T. Budd, M. J. Ellis, A. Stopeck, J. Matera, M. C. Miller, J. M. Reuben, G. V. Doyle, W. J. Allard, L. W. Terstappen, D. F. Hayes, N. Engl. J. Med. 351, 781 (2004); S. Mocellin, D. Hoon, A. Ambrosi, D. Nitti, C. R. Rossi, Clin. Cancer Res. 12, 4605 (2006); G. Wiedswang, B. Naume, Nat. Clin. Pract. Oncol. 4, 154 (2007)). The effectiveness of therapeutic treatments can also be measured by the number of CTCs in blood (F. Nole, E. Munzone, L. Zorzino, I. Minchella, M. Salvatici, E. Botteri, M. Medici, E. Verri, L. Adamoli, N. Rotmensz, A. Goldhirsch, M. T. Sandri, Ann. Oncol. 19, 891 (2008); A Rolle, R. Gunzel, U. Pachmann, B. Willen, K. Hoffken, K. Pachmann, World J. Surg. Oncol. 3, 18 (2005)). Thus, there is much interest in isolating, quantifying and studying these cells obtained from peripheral blood. CTCs are of very low concentration in blood which poses the technical difficulty of detecting these rare cells (J. E. Losanoff, W. Zhu, W. Qin, F. Mannello, E. R. Sauter, Breast 17, 540 (2008); V. Zieglschmid, C. Hollmann, O. Bocher, Crit. Rev. Clin. Lab. Sci. 42, 155 (2005)).

The absolute number of CTCs in blood of cancer patients varies and depends on the conditions of the patients. Leading techniques to enumerate CTCs include immuno-magnetic separation followed by immunocytochemistry detection, such as the CellSearch® system sold by Veridex LLC (a Johnson & Johnson company) of Raritan, N.J., U.S.A. (M. Cristofanilli, G. T. Budd, M. J. Ellis, A. Stopeck, J. Matera, M. C. Miller, J. M. Reuben, G. V. Doyle, W. J. Allard, L. W. Terstappen, D. F. Hayes, N. Engl. J. Med. 351, 781 (2004); H. Yagata, S. Nakamura, M. Toi, H. Bando, S. Ohno, A. Kataoka, Int J Clin Oncol 13, 252 (2008)). A further leading technique is RT-PCR to indicate the presence of CTCs in peripheral blood (L. A. Mattano Jr., T. J. Moss, S. G. Emerson, Cancer Res. 52, 4701 (1992); C. P. Schroder, M. H. Ruiters, S. de Jong, A. T. Tiebosch, J. Wesseling, R. Veenstra, J. de Vries, H. J. Hoekstra, L. F. de Leij, E. G. de Vries, Int. J. Cancer 106, 611 (2003)). These methods have been successfully demonstrated on various cancer types (S. Dawood, K. Broglio, V. Valero, J. Reuben, B. Handy, R. Islam, S. Jackson, G. N. Hortobagyi, H. Fritsche, M. Cristofanilli, Cancer 113, 2422 (2008); R. Szatanek, G. Drabik, J. Baran, P. Kolodziejczyk, J. Kulig, J. Stachura, M. Zembala, Oncol. Rep. 19, 1055 (2008); C. S. Wong, M. T. Cheung, B. B. Ma, E. Pun Hui, A. C. Chan, C. K. Chan, K. C. Lee, W. Cheuk, M. Y. Lam, M. C. Wong, C. M. Chan, J. K. Chan, and A. T. Chan, Int. J. Surg. Pathol. 16, (2008)). Alternative methodologies such as a direct visualization assay (H. J. Kahn, A. Presta, L. Y. Yang, J. Blondal, M. Trudeau, L. Lickley, C. Holloway, D. R. McCready, D. Maclean, A. Marks, Breast Cancer Res. Treat. 86, 237 (2004)), fluorescent activated cell sorter (FACS) (J. G. Moreno, S. M. O'Hara, S. Gross, G. Doyle, H. Fritsche, L. G. Gomella, L. W. Terstappen, Urology 58, 386 (2001)), fibre-optic array scanning technology (FAST) cytometer (R. T. Krivacic, A. Ladanyi, D. N. Curry, H. B. Hsieh, P. Kuhn, D. E. Bergsrud, J. F. Kepros, T. Barbera, M. Y. Ho, L. B. Chen, R. A. Lerner, R. H. Bruce, Proc. Natl. Acad. Sci. USA 101, 10501 (2004)) and anti-EpCAM coated microfabricated structures (S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature 450, 1235 (2007)) have also been used to enumerate CTCs in blood samples. Complex procedures, tedious inspections and long processing time are the limiting factors associated with most existing techniques. Furthermore, viability of the isolated cells are lost as fixing of the samples is required by most existing techniques.

There is much to understand about the condition of CTCs whilst in circulation (K. Pantel. R. H. Brakenhoff, B. Brandt, Nat. Rev. Cancer 8, 329 (2008)) and having viable cells after isolation would allow studies to be carried out on CTC sub-populations. This may provide valuable insights to the biological characteristics of the disease such as the link between cancer stem cells and metastasis. Although recent work isolated CTCs (S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature 450, 1235 (2007)), the isolated cells are likely to be difficult to retrieve due to the binding of the tumor associated antigens to the device. Retrieving these cells may require high mechanical forces or biochemical agents and the integrity of these cells might be affected as a result (S. F. Chang, C. A. Chang, D. Y. Lee, P. L. Lee, Y. M. Yeh, C. R. Yeh, C. K. Cheng, S. Chien, J. J. Chiu, Proc. Natl. Acad. Sci. USA 105, 3927 (2008)). In addition, most methodologies will require functional modifications which is less desirable (O. Lara, X. Tong, M. Zborowski, J. J. Chalmers, Exp. Hematol. 32, 891 (2004)).

Whilst existing techniques facilitate the identification of cells from within a sample, they each have their own shortcomings.

Accordingly, it is desired to provide an improved technique for identifying cells from within a sample.

### SUMMARY

According to a first aspect, there is provided a micro-fluidic device operable to isolate target cells from a biological fluid, the micro-fluidic device comprising: an inlet operable to receive the biological fluid, the biological fluid comprising target cells and other components; an waste outlet operable to receive at least the other components of the biological fluid; a plurality of parallel arrays of cell isolation wells coupling the inlet with the waste outlet, each parallel array of cell isolation wells supporting a flow of the biological fluid from the inlet to the waste outlet in response to a pressure differential thereacross, each array of cell isolation wells comprising a plurality isolation wells, each isolation well being dimensioned to mechanically trap the target cells therein whilst permitting flow of other components of the biological fluid; and at least one pressure maintenance structure operable to assist in maintaining a predetermined pressure differential across each of the plurality of parallel arrays of cell isolation wells.

The first aspect recognises that there are practical difficulties when attempting to isolate target cells from within a test sample. For example, there are difficulties relating to the physical properties of the test sample being processed and difficulties encountered when attempting to maximise the number of target cells being isolated. For example, because the concentration of target cells to be isolated is typically very low, it may be necessary to increase the volume of sample being processed or increase the efficiency of target cell isolation using, for example, cell isolation structures, wells or traps. Processing an increased volume of sample can take more time. Although the rate of target cell isolation can be increased by placing more cell isolation structures in series to increase the likelihood of a target cell being retained, this typically requires operating at an increased pressure due to the increased resistance to flow. Likewise, whilst it may be possible to place an increased number of cell isolation structures in parallel to decrease resistance to flow, this causes difficulty in maintaining a consistent pressure differential across the increased number of cell isolation structures to keep the cells isolated therein.

Accordingly, a micro-fluidic device operable to isolate or capture target material, such as cells, from a fluid, such as a biological fluid, is provided. A sample inlet may receive the biological fluid which may contain target cells and other components. A waste outlet may be provided which receives at least some of the other components of the biological fluid. Coupling the sample inlet with the waste outlet may be a plurality of parallel arrays of isolation wells. Each of the parallel arrays may enable fluid to flow from the sample inlet to the waste outlet following the application of a pressure differential across the arrays of isolation wells. Each array may comprise a number of isolation wells and each isolation well may be dimensioned to physically trap the target cells. The arrangement of the isolation wells may be such that the likelihood of capturing the other components of the fluid may be reduced. One or more pressure maintenance structures may be provided which facilitates achieving a pressure differential within the device when the device is operated.

In this way, it can be seen that a device is provided which may provide an increased number of parallel arrays of isolation wells to enable an increased volume of fluid to be processed using an increased number of traps in order to increase the number of target cells which are retained. By providing the arrays in parallel, the volume of sample that can be processed per unit time may be increased. This may reduce the processing time of a sample whilst increasing the efficiency of target cell isolation. Also, by using pressure maintenance structures, each of the parallel arrays of cell isolation structures may be operated effectively to ensure correct processing of the sample. In particular, the likelihood of the flow of the sample through each of the parallel arrays of traps being maintained in a controllable manner may be increased. This helps to ameliorate the problem of increasing the scale of the micro-fluidic device which is difficult because it is difficult to reliably achieve a predictable flow in the presence of samples having wide-varying characteristics. Such an approach may provide an enhanced degree of trapping in a reduced amount of time. Also, this facilitates scaling up the device and may ameliorate issues of the workings of the system in a micro-fluidic environment.

In one embodiment, the at least one pressure maintenance structure comprises: a primary pre-filter array positioned between the inlet and the plurality of parallel arrays of cell isolation wells, the primary pre-filter array comprising a plurality of rows of primary structures dimensioned to mechanically trap bodies within the biological fluid larger than the target cells. A problem with some test samples, such as, for example, blood, is that the sample may be of poor quality and may be subject to coagulation. Accordingly, the sample may cause local blockages or disrupt the flow within the arrays of cell isolation wells. Such disruption in flow may prevent a pressure differential from being maintained across cell isolation wells. Accordingly, a pre-filter array may be positioned between the sample inlet and the arrays of cell isolation wells. The pre-filter array may comprise structures which are configured to trap components within the sample fluid having predetermined dimensions. The provision of such a pre-filter array may enable loose material to be held. By providing the pre-filter array prior to the cell isolation wells, the materials can be confined outside of the main micro-fluidic structure which enables the dimensions of subsequent micro-fluidic structures to be scaled down considerably, thereby facilitating a reduction in size of the micro-fluidic device. It will be appreciated that the area presented by the pre-filter array may determine how much material can be retained. Likewise, it will be appreciated that a distance between each primary structure may affect the amount of material which is able to pass through the pre-filter array. In addition, such a pre-filter array may be used to trap emboli which may also contain CTCs. These may be stained and retrieved via a debris removal outlet without passing through the cell isolation wells.

In one embodiment, adjacent rows have primary structures which are offset with respect to each other. Offsetting rows of primary structures provides a mechanically robust array which is an effective filter, since large material is likely to have its path obstructed by such offset structures.

In one embodiment, each primary structure presents a width to the flow of the biological fluid and a distance between adjacent primary structures in a row is approximately the width.

In one embodiment, the distance between adjacent primary structures in each row decreases.

In one embodiment, the distance between adjacent primary structures decreases from around 100 μm to 30 μm.

In one embodiment, the width presented by primary structures in each row decreases.

In one embodiment, a distance between adjacent rows is greater than the width.

In one embodiment, each primary structure has a generally curved cross-section. Providing a generally curved cross-section helps to reduce any flow impedance through the pre-filter array. In addition, a curved or round cross-section reduces additional stresses on any target cells within the fluid passing through the pre-filter array.

In one embodiment, each primary structure comprises a cylindrical pillar. In one embodiment, each primary structure has a radius of approximately 20 μm, the distance between adjacent primary structures in a row is approximately 20 μm and the distance between adjacent rows is approximately 40 μm. Of course, it will be appreciated that the dimensions of components the pre-filter array will typically be determined based on the size of the target cells and the likely size of large material which needs to be trapped, determined by properties of the fluid being analysed.

In one embodiment, the primary pre-filter array is positioned away from the plurality of parallel arrays of cell isolation wells by a predetermined distance. Positioning the pre-filter array away from the array of cell isolation wells helps to restore uniform flow and may equalize the pressure across the arrays of cell isolation wells. In other words, the effect of any local obstructions within the pre-filter array on the flow of material through each of the arrays of cell isolation wells can be reduced by providing a buffer region between the pre-filter array and the arrays of cell isolation wells to equalize any local pressure variations.

In one embodiment, the primary pre-filter array comprises an inlet flow distributor positioned between the inlet and the plurality of rows of primary structures, the inlet flow distributor comprising a tree of fluidic channels. Accordingly, a flow distributor structure may be provided between the inlet and the primary structures. Such a flow distributor distributes the biological fluid to the plurality of rows of primary structures to provide an even pressure distribution across the rows of primary structures and to obviate the effects of local flow disturbances caused by clogging. The greater the number of levels within the tree, the smoother the transition. It will also be appreciated that providing such an inlet flow distributor may become necessary as the device is scaled up to include many numbers of parallel primary pre-filter arrays and therefore helps to provide for unfettered scalability.

In one embodiment, a root level of the tree presents a number of fluidic channels to the inlet and a leaf level of the tree presents ‘n’ fluidic channels to the plurality of rows of primary structures. It will be appreciated that the number of fluidic channels presented to the plurality of rows of primary structures need not necessarily exactly match the number of primary structures in the first row of the plurality of rows of primary structures.

In one embodiment, each leaf level away from the root level and towards the plurality of rows of primary structures presents at least an additional fluidic channel.

In one embodiment, the primary pre-filter array comprises a flow combiner positioned between the plurality of rows of primary structures and the plurality of arrays of cell isolation wells, the flow combiner comprising a tree of fluidic channels.

In one embodiment, the micro-fluidic device comprises a collection outlet operable to receive debris trapped by the pre-filter array.

In one embodiment, the at least one pressure maintenance structure comprises: a secondary pre-filter array positioned between the primary pre-filter array and the plurality of parallel arrays of cell isolation wells, the secondary pre-filter array comprising at least one row of secondary structures dimensioned to mechanically trap bodies within the biological fluid larger than the target cells. Accordingly, a secondary pre-filter array may be provided positioned between the arrays of cell isolation wells and the primary pre-filter array. Such a secondary array may enhance the ability to pre-filter the fluid.

In one embodiment, each secondary structure presents a width to the flow of the biological fluid and a distance between adjacent secondary structures in a row is less than the width.

In one embodiment, the secondary pre-filter array comprises a plurality of rows of secondary structures.

In one embodiment, the distance between adjacent secondary structures in each row decreases.

In one embodiment, each secondary structure has a generally rectilinear cross-section. By providing a secondary structure presenting a generally flat area to the arrays of cell isolation wells an increased resistance to any flow from the cell isolation wells to the pre-filter array may be provided. Such an arrangement may help to prevent backflow to the inlet during subsequent processing, such as, for example, flushing or staining or retrieving target cells.

In one embodiment, each secondary structure is has a width of approximately 35 μm and the distance between adjacent secondary structures in a row is approximately 20 μm. It will be appreciated that, once again, the exact dimensions of the secondary structure will need to enable target cells to pass through whilst preventing the passage of any larger bodies into the subsequent structures.

In one embodiment, the row of secondary structures is partitioned by supporting structures positioned to align with each boundary of the plurality of parallel arrays of cell isolation wells. Providing supporting structures helps increase the mechanical integrity of the secondary pre-filter array. By aligning the secondary structures with the boundaries of the arrays of cell isolation wells, the flow of material from the pre-filter array may be aligned with the arrays of cell isolation wells, thereby improving flow.

In one embodiment, the secondary pre-filter array is positioned away from the plurality of parallel arrays of cell isolation wells by a predetermined distance. Again, by positioning the secondary structures away from the arrays of cell isolation wells provides a buffer region within which pressure equalization can occur despite local obstructions being present.

In one embodiment, the predetermined distance is approximately 500 μm. Of course, it will be appreciated that the distance between the pre-filter array and the array of cell isolation wells will depend on the characteristics of the fluid being processed and the dimensions of the device.

In one embodiment, the at least one pressure maintenance structure comprises: a flow combiner positioned between the plurality of parallel arrays of cell isolation wells and the waste outlet and, the flow combiner comprising a tree of fluidic channels operable to maintain a uniform pressure presented by the outlet to each of the plurality of parallel arrays of cell isolation wells. Accordingly, a flow combination structure may be provided between the arrays of cell isolation wells and the waste outlet. Without such a combiner, the pressure experienced by each of the arrays of cell isolation wells may vary considerably, dependent on the relative location of the waste outlet to that array of cell isolation wells. For example, if each of the arrays of cell isolation wells were connected directly to the waste outlet, then those physically closest to the waste outlet would be presented with a lower pressure than those physically located relatively further away. This may lead to disruptions in flow within arrays of cell isolation wells and may result in the flow of one array of cell isolation wells interfering with the flow of another. By providing a flow combiner, a more even distribution of pressure across each level of the tree of fluidic channels is provided. The greater the number of levels within the tree, the smoother the transition is to the waste outlet. It will also be appreciated that providing such a flow combiner may become necessary as the device is scaled up to include many numbers of parallel arrays of isolation wells and therefore helps to provide for unfettered scalability.

In one embodiment, a root level of the tree presents one fluidic channel to the waste outlet and a leaf level of the tree presents ‘n’ fluidic channels to the plurality of parallel arrays of cell isolation wells. It will be appreciated that the number of fluidic channels presented to the arrays of isolation wells need not necessarily exactly match the number of arrays of cell isolation wells.

In one embodiment, each leaf level away from the root level and towards the plurality of parallel arrays of cell isolation wells presents an additional fluidic channel.

In one embodiment, the fluidic channels have a width of approximately 150 μm. It will be appreciated that the exact dimensions of the flow combiner will depend on the characteristics of the sample and the dimensions of the device.

In one embodiment, the micro-fluidic device comprises: a target cell recovery outlet and in which the at least one pressure maintenance structure comprises: a conduit coupling the plurality of parallel arrays of cell isolation wells with the target cell recovery outlet, the conduit being shaped to restrict flow of fluid therethrough. Accordingly, an outlet may be provided through which target cells may be recovered. It will be appreciated that the target cells may be recovered from the arrays of cell isolation wells and so the target cell recovery outlet will typically need to be coupled with those arrays of cell isolation wells. In order to prevent the presence of the target cell recovery outlet from disrupting the flow through the arrays of cell isolation wells, a conduit which restricts flow from the arrays of cell isolation wells to the target cell recovery outlet may be provided. Once again, it will be appreciated that this facilitates the scaling of the device to include many more arrays of cell isolation wells.

In one embodiment, the conduit is shaped to restrict flow of fluid by causing at least one change in direction of flow. Although a change in direction of flow may be utilised to restrict the flow of fluid, it will be appreciated that other mechanisms for restricting flow could equally be used.

In one embodiment, the conduit is shaped to cause at least two orthogonal changes in direction of flow.

In one embodiment, the conduit is shaped to cause at least four orthogonal changes in direction of flow.

In one embodiment, each section of the conduit has a constant width.

In one embodiment, the conduit has a width of approximately 50 μm. Of course, it will be appreciated that the exact dimensions of the conduits will typically depend on the size of the target cells which need to be recovered.

In one embodiment, each array of cell isolation wells comprises a plurality of subarrays of cell isolation wells spaced apart in a direction of the flow and a plurality of the conduits are provided, each conduit coupling a sub-array with the target cell recovery outlet. Accordingly, rather than providing just one conduit for recovering the target cells from an array of cell isolation wells, it is possible to split the array of cell isolation wells into subarrays and provide a conduit for each of those sub-arrays. It will be appreciated that this considerably facilitates the recovery of target cells from the array of cell isolation wells.

In one embodiment, each conduit is received by a common conduit coupled with the target cell recovery outlet.

In one embodiment, the waste outlet and the target cell recovery outlet are operable to maintain a pressure differential to cause a change in direction of flow within the parallel arrays of cell isolation wells to release trapped cells to be conveyed to the target cell recovery outlet.

In one embodiment, the at least one pressure maintenance structure comprises: a flow distributor coupling the plurality of parallel arrays of cell isolation wells with the inlet, the distributor comprising a tree of fluidic channels. Once again, it will be appreciated that this facilitates the scaling of the device to include many more arrays of cell isolation wells. The flow distributor may be provided between the inlet or any filter and the arrays of cell isolation wells. Such a flow distributor distributes the biological fluid to the arrays of cell isolation wells to provide an even pressure distribution across the arrays of cell isolation wells and to obviate the effects of local flow disturbances caused by clogging. The greater the number of levels within the tree, the smoother the transition. It will also be appreciated that providing such an inlet flow distributor may become necessary as the device is scaled up to include many numbers of parallel primary pre-filter arrays and therefore helps to provide for unfettered scalability.

In one embodiment, a root level of the tree presents a number of fluidic channels to the inlet and a leaf level of the tree presents ‘n’ fluidic channels to the arrays of cell isolation wells. It will be appreciated that the number of fluidic channels presented to the arrays of cell isolation wells need not necessarily exactly match the number of arrays of cell isolation wells.

In one embodiment, each leaf level away from the root level and towards the plurality of arrays of cell isolation wells presents at least an additional fluidic channel.

In one embodiment, the micro-fluidic device comprises: a target cell recovery outlet and the flow distributor couples the plurality of parallel arrays of cell isolation wells with the target cell recovery outlet. Accordingly, an outlet may be provided through which target cells may be recovered. It will be appreciated that the target cells may be recovered from the arrays of cell isolation wells and so the target cell recovery outlet will typically need to be coupled with those arrays of cell isolation wells. In order to facilitate the target cell recovery, the flow distributor provides a uniform flow field for efficient cell collection. Once again, it will be appreciated that this facilitates the scaling of the device to include many more arrays of cell isolation wells. Such an arrangement, when the flow is reversed, helps to concentrate target cells recovered from the arrays of cell isolation wells when being provided to the target cell recovery outlet.

In one embodiment, the target cell recovery outlet is located on the inlet side of the plurality of parallel arrays of cell isolation wells.

In one embodiment, the micro-fluidic device comprising: a reagent inlet operable to receive a reagent, the reagent inlet comprising a conduit operable to deliver the reagent to between the inlet and the plurality of parallel arrays of cell isolation wells. Accordingly, a reagent can be added to react with the contents of the micro-fluidic device.

In one embodiment, the conduit is operable to deliver the reagent to between primary pre-filter array and the secondary pre-filter array. Accordingly, a reagent can be added between the pre-filter array and the arrays of cell isolation wells in order to avoid any clogging within the pre-filter array and to enable the reagent to reach the target cells much more quickly.

In one embodiment, the reagent comprises at least one fluorescence in situ hybridization reagent.

In one embodiment, the reagent comprises at least one of a first fluorescent probe and a second fluorescent probe.

In one embodiment, a primary pre-filter array is provided between the reagent inlet and the plurality of parallel arrays of cell isolation wells.

In one embodiment, each array of cell isolation wells is coupled in parallel between the inlet and the outlet.

In one embodiment, the cell isolation structures comprise crescent-shaped structures operable to retain target cells such as, for example, cancer cells, fetal cells or stem cells.

In one embodiment, each cell isolation structure defines at least one gap in the crescent-shaped structure.

In one embodiment, each gap is around 6 μm to 9 μm.

In one embodiment, the crescent-shaped structures are tilted with a tilt axis which is non-orthogonal with respect to a direction of flow through the array of cell isolation wells.

In one embodiment, each cell isolation well within each row of cell isolation wells is tilted with the same tilt axis.

In one embodiment, each cell isolation well within adjacent rows of cell isolation wells is tilted with opposing tilt axes.

In one embodiment, cell isolation wells within each row are spaced apart by gaps and cell isolation wells within adjacent rows are positioned to align with the gaps.

In one embodiment, a distance between adjacent rows of cell isolation wells increases in a direction of flow from the inlet to the outlet.

According to a second aspect, there is provided a method of isolating target cells from a biological fluid, the method comprising the steps of: receiving the biological fluid at an inlet, the biological fluid comprising target cells and other components; applying a pressure differential across a plurality of parallel arrays of cell isolation wells coupling the inlet with a waste outlet operable to receive at least the other components of the biological fluid, each parallel array of cell isolation wells supporting a flow of the biological fluid from the inlet to the waste outlet in response to a pressure differential thereacross, each array of cell isolation wells comprising a plurality isolation wells, each isolation well being dimensioned to mechanically trap the target cells therein whilst permitting flow of other components of the biological fluid; and maintaining a predetermined pressure differential across each of the plurality of parallel arrays of cell isolation wells.

In one embodiment, the step of maintaining a predetermined pressure differential comprises the step of: positioning a primary pre-filter array between the inlet and the plurality of parallel arrays of cell isolation wells, the primary pre-filter array comprising a plurality of rows of primary structures dimensioned to mechanically trap bodies within the biological fluid larger than the target cells.

In one embodiment, adjacent rows have primary structures which are offset with respect to each other.

In one embodiment, each primary structure presents a width to the flow of the biological fluid and a distance between adjacent primary structures in a row is approximately the width.

In one embodiment, the distance between adjacent primary structures in each row decreases.

In one embodiment, the distance between adjacent primary structures decreases from around 100 μm to 30 μm.

In one embodiment, the width presented by primary structures in each row decreases.

In one embodiment, each primary structure has a depth and a distance between adjacent rows is greater than the width.

In one embodiment, each primary structure has a generally curved cross-section.

In one embodiment, each primary structure comprises a cylindrical pillar.

In one embodiment, each primary structure is has a radius of approximately 20 μm, the distance between adjacent primary structures in a row is approximately 20 μm and the a distance between adjacent rows is approximately 40 μm.

In one embodiment, the step of positioning comprises: positioning the primary prefilter array away from .the plurality of parallel arrays of cell isolation wells by a predetermined distance.

In one embodiment, the primary pre-filter array comprises an inlet flow distributor positioned between the inlet and the plurality of rows of primary structures, the inlet flow distributor comprising a tree of fluidic channels.

In one embodiment, a root level of the tree presents a number of fluidic channels to the inlet and a leaf level of the tree presents ‘n’ fluidic channels to the plurality of rows of primary structures.

In one embodiment, each leaf level away from the root level and towards the plurality of rows of primary structures presents at least an additional fluidic channel.

In one embodiment, the primary pre-filter array comprises a flow combiner positioned between the plurality of rows of primary structures and the plurality of arrays of cell isolation wells, the flow combiner comprising a tree of fluidic channels.

In one embodiment, the method comprises the step of applying a pressure differential to receive debris trapped by the pre-filter array at a collection outlet.

In one embodiment, the step of maintaining a predetermined pressure differential comprises the step of: positioning a secondary pre-filter array between the primary pre-filter array and the plurality of parallel arrays of cell isolation wells, the secondary pre-filter array comprising at least one row of secondary structures dimensioned to mechanically trap bodies within the biological fluid larger than the target cells.

In one embodiment, each secondary structure presents a width to the flow of the biological fluid and a distance between adjacent secondary structures in a row is less than the width.

In one embodiment, the secondary pre-filter array comprises a plurality of rows of secondary structures.

In one embodiment, the distance between adjacent secondary structures in each row decreases.

In one embodiment, each secondary structure has a generally rectilinear cross-section.

In one embodiment, each secondary structure is has a width of approximately 35 μm and the distance between adjacent secondary structures in a row is approximately 20 μm.

In one embodiment, the method comprises the step of: partitioning the row of secondary structures by supporting structures positioned to align each boundary of the plurality of parallel arrays of cell isolation wells.

In one embodiment, the step of positioning comprises: positioning the secondary prefilter array away from the plurality of parallel arrays of cell isolation wells by a predetermined distance.

In one embodiment, the predetermined distance is approximately 500 μm.

In one embodiment, the step of maintaining a predetermined pressure differential comprises the step of: positioning a flow combiner between the plurality of parallel arrays of cell isolation wells and the waste outlet and, the flow combiner comprising a tree of fluidic channels operable to maintain a uniform pressure presented by the outlet to each of the plurality of parallel arrays of cell isolation wells.

In one embodiment, a root level of the tree presents one fluidic channel to the waste outlet and a leaf level of the tree presents ‘n’ fluidic channels to the plurality of parallel arrays of cell isolation wells.

In one embodiment, each leaf level away from the root level and towards the plurality of parallel arrays of cell isolation wells presents an additional fluidic channel.

In one embodiment, the fluidic channels have a width of approximately 150 μm.

In one embodiment, the step of maintaining a predetermined pressure differential comprises the step of: providing a conduit coupling the plurality of parallel arrays of cell isolation wells with a target cell recovery outlet, the conduit being shaped to restrict flow of fluid therethrough.

In one embodiment, the method comprises the step of recovering target cells from the target cell recovery outlet.

In one embodiment, the step of recovering target cells from the target cell recovery outlet comprises reversing a flow through the cell isolation wells by applying a differential pressure between the waste outlet and the target cell recovery outlet.

In one embodiment, the conduit is shaped to restrict flow of fluid by causing at least one change in direction of flow.

In one embodiment, the conduit is shaped to cause at least two orthogonal changes in direction of flow.

In one embodiment, the conduit is shaped to cause at least four orthogonal changes in direction of flow.

In one embodiment, each section of the conduit has a constant width.

In one embodiment, the conduit has a width of approximately 50 μm.

In one embodiment, each array of cell isolation wells comprises a plurality of subarray of cell isolation wells spaced apart in a direction of the flow and a plurality of the conduits are provided, each conduit coupling a sub-array with the target cell recovery outlet.

In one embodiment, each conduit is received by a common conduit coupled with the target cell recovery outlet.

In one embodiment, the step of recovering target cells from the target cell recovery outlet comprises reversing a flow through the cell isolation wells by maintaining a pressure differential between the waste outlet and the target cell recovery outlet to cause a change in direction of flow within the parallel arrays of cell isolation wells to release trapped cells to be conveyed to the target cell recovery outlet.

In one embodiment, the step of maintaining a predetermined pressure differential comprises the step of: providing a flow distributor coupling the plurality of parallel arrays of cell isolation wells with the inlet, the flow distributor comprising a tree of fluidic channels.

In one embodiment, a root level of the tree presents a number of fluidic channels to the inlet and a leaf level of the tree presents ‘n’ fluidic channels to the arrays of cell isolation wells.

In one embodiment, each leaf level away from the root level and towards the plurality of arrays of cell isolation wells presents at least an additional fluidic channel.

In one embodiment, the method comprises the step of providing a target cell recovery outlet, the flow distributor coupling the plurality of parallel arrays of cell isolation wells with the target cell recovery outlet.

In one embodiment, the method comprises the step of recovering target cells from the target cell recovery outlet.

In one embodiment, the step of recovering target cells from the target cell recovery outlet comprises reversing a flow through the cell isolation wells be applying a differential pressure between one of a buffer port and the waste outlet and the target cell recovery outlet.

In one embodiment, the method comprises locating the target cell recovery outlet on the inlet side of the plurality of parallel arrays of cell isolation wells.

In one embodiment, the method comprises the step of: receiving a reagent at a reagent inlet, the reagent inlet comprising a conduit operable to deliver the reagent to between the inlet and the plurality of parallel arrays of cell isolation wells.

In one embodiment, the conduit is operable to deliver the reagent to between primary pre-filter array and the secondary pre-filter array.

In one embodiment, the reagent comprises at least one fluorescence in situ hybridization reagent.

In one embodiment, the reagent comprises at least one of a first fluorescent probe and a second fluorescent probe.

In one embodiment, a primary pre-filter array is provided between the reagent inlet and the plurality of parallel arrays of cell isolation wells.

In one embodiment, each array of cell isolation wells is coupled in parallel between the inlet and the outlet. In one embodiment, the cell isolation structures comprise crescent-shaped structures operable to retain target cells such as, for example, cancer cells, fetal cells or stem cells. In one embodiment, the cell isolation structures comprise ‘U’ or ‘V’-shaped structures.

In one embodiment, each cell isolation structure defines at least one gap in the crescent-shaped structure.

In one embodiment, each gap is around 6 μm to 9 μm.

In one embodiment, the crescent-shaped structures are tilted with a tilt axis which is non-orthogonal with respect to a direction of flow through the array of cell isolation wells.

In one embodiment, each cell isolation well within a row of cell isolation wells is tilted with the same tilt axis.

In one embodiment, each cell isolation well within adjacent rows of cell isolation wells is tilted with opposing tilt axes.

In one embodiment, cell isolation wells within each row are spaced apart by gaps and cell isolation wells within adjacent rows are positioned to align with the gaps.

In one embodiment, a distance between adjacent rows of cell isolation wells increases in a direction of flow from the inlet to the outlet.

In one embodiment, the method comprises the step of performing fluorescence in situ hybridization (FISH). In will be appreciated that this may be performed on cells within the isolation wells or the cells may be transferred to another structure within or outside the device to perform FISH.

In one embodiment, the method comprises the step of performing a secondary fluorescence in situ hybridization (FISH). In this way, it is possible to study sub-populations of cells because the cells are immobilised and it is possible to determine which cells are expressing which proteins.

Further particular and preferred aspects of the present invention are set out in the accompanying independent and dependent claims. Features of the dependent claims may be combined with the features of the independent claims as appropriate, and in combinations other than those explicitly set out in the claims.

The practice of the present invention will employ, unless otherwise indicated, conventional techniques of biochemistry, cell and molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0- 87969-630-3. Each of these general texts is herein incorporated by reference.

### BRIEF DESCRIPTION OF THE FIGURES

Embodiments of the present invention will now be described further, with reference to the accompanying drawings, in which:

FIG. 1 illustrates a diagnostic system incorporating a micro-fluidic device according to one embodiment;

FIG. 2 shows a laboratory prototype of a fabricated micro-fluidic device;

FIG. 3 illustrates an arrangement of the micro-fluidic device according to one embodiment in more detail;

FIG. 4A illustrates a configuration of a first pre-filter array in more detail;

FIG. 4B illustrates a configuration of a secondary pre-filter in more detail;

FIG. 4C illustrates an arrangement of a plurality of arrays of cell isolation wells in more detail;

FIG. 4D illustrates a configuration of a gradient generator in more detail;

FIG. 4E illustrates a configuration of flow restrictors in more detail;

FIG. 5 illustrates cell isolation wells in more detail;

FIG. 6 illustrates an arrangement of the micro-fluidic device according to another embodiment in more detail;

FIG. 7A, FIG. 7B and FIG. 7C illustrate a flow combiner in more detail;

FIG. 8 illustrates a pre-filter array in more detail; and

FIG. 9 illustrates cell isolation wells in more detail.

### DETAILED DESCRIPTION

A description of example embodiments of the invention follows. Cancer metastasis is the main attribute to cancer-related deaths. Furthermore, clinical reports have shown a strong correlation between disease development and the number of circulating tumor cells (CTCs) in the peripheral blood of cancer patients. In accordance with embodiments, there is provided a label-free micro-fluidic device capable of isolating target cells, such as cancer cells or other cells, from whole blood via their distinctively different physical properties such as deformability and size. The isolation efficiency is on average at least 80% for tests performed on breast cancer, colon cancer and other types of cancer cell lines. Viable isolated cells are also obtained which may give further insights to enhance the understanding of the metastatic process.

Contrasting with conventional biochemical techniques, a micro-fluidic device according to embodiments provides a mechanistic and efficient means of isolating viable cancer cells in blood. The micro-fluidic device has the potential to be used for routine monitoring of cancer development and cancer therapy in a clinical setting. In accordance with embodiments, there is provided a device that presents a means to enumerate and isolate viable CTCs or other target cells from peripheral blood with a high throughput and high efficiency. Isolation and quantification of CTCs presents an alternate disease marker that assists in monitoring cancer progression and determining overall survival. In accordance with embodiments, retrieving viable CTCs assists in the further study of key biological determinants to the disease which might provide insights to the lethality of the disease. Compared with most existing methodologies, the ability to separate CTCs or other target cells from blood in fewer steps in accordance with embodiments reduces the complexity of obtaining vital disease information. Most existing methodologies require multiple enrichment and identification procedures which are tedious and time consuming. A device in accordance with embodiments is able to take whole blood samples for processing directly. Furthermore, viability of the cells is compromised in most existing approaches. Even in existing techniques using anti-EpCAM coated microstructures that are able to isolate viable CTCs, retrieval is a challenge because high shear flows are required to break the affinity of the biochemical bonds. In addition, EpCAM negative CTCs will be missed.

Micro-fluidic devices provide an alternative technique compared to conventional biochemical separations. Devices utilizing dielectrophoretic forces to separate and manipulate cells are advantageous as they do not require functionalization of the sample or the microfluidic device (P. Y. Chiou, A. T. Ohta, M. C. Wu, Nature 436, 370-372 (2005); D. S. Gray, J. L. Tan, J. Voldman, C. S. Chen, Biosens. Bioelectron. 19, 771-780 (2004); A. Rosenthal, J. Voldman, Biophys. J. 88, 2193-2205 (2005); J. Voldman, Curr Opin Biotechnol 17, (2006)). However, efficient cancer cell separation may be difficult due to the low concentration of CTCs or other target cells in blood and the relative similar dimensions of leukocytes with cancer cells.

In accordance with embodiments, through a biomechanical means of isolation that utilizes CTCs' or other target cells distinct biorheological difference with blood constituents, the retrieval of CTCs or other target cells is made relatively easy to achieve by controlling the input/output conditions. In addition, no functional biochemical modification of the device or CTCs or other target cells is necessary to maintain the integrity of CTCs or other target cells. The microsystem of embodiments has a high throughput and is able to process the blood samples within minutes. Further, embodiments permit real time visualization of the isolation process. Further, a system may provide a simple means of enumerating target cells (such as CTCs) using a uniform array. In addition, a device may permit real time enumeration of isolated cells.

For example, embodiments enable fluorescence in situ hybridization (FISH). Such a technique can be used to detect and localise the presence or absence of specific DNA sequences on chromosomes of targets cells. Fluorescent probes are introduced via the reagent inlet 150 that bind to only those parts of the chromosome with which they show a high degree of sequence similarity. Fluorescence microscopy can be used to find out where the fluorescent probe is bound to the chromosomes. FISH can also be used to detect and localize specific mRNAs within samples and can help define the spatial-temporal patterns of gene expression within cells and tissues. In embodiments, FISH involves fixing the cells (i.e., killing the cells and chemically cross-linking proteins, nucleic acids), exposing the fixed cells to a staining agent and visualising it. The staining agent typically is a nucleic acid probe which binds to portions of the DNA and has a fluorescent tag so the chromosomes can be visualised. The micro-fluidic device 10 enables target cells to be sieved, immobilised in the isolation wells 190 and then visualised or stained to identify target cells in a first round. For example, a stain that picks out tumour cells may be used (e.g., an antibody that binds to a tumour specific antigen expressed on the cell surface) to see which cells that have been sieved are tumour cells. A second round of staining using FISH can then be used to look at the chromosomes of those particular cells, (e.g., to see if there are chromosomal breaks that might be associated with the tumour phenotype). Hence, double staining is possible. Because the target cells are immobilised in the isolation wells 190 in which they were caught during the sieving, increased certainty is possible that whatever cells were identified using the first stain are the cells which are being examined after the second stain. The FISH signals from those positions in the matrix which are positive from the first signal can simply be examined. Without the immobilisation, it is not possible to do this. FISH may be performed either on the target cells within the isolation wells 190 or may be performed within a FISH structure (not shown) on the microfluidic device 10 or outside the microfluidic device 10. For example, a separate channel (not shown) may be provided on the side of the microfluidic device 10 (having appropriate couplings for introduction of the reagents) within which FISH may be performed or alternatively or additionally a structure (not shown) may be provided which enables small amounts of reagents to be located near the isolation wells and piped thereto so that minimal reagents are used. Depending on the staining used, additional inlet ports and tubing may be required.

A device in accordance embodiments has potential application to aid clinicians in cancer disease monitoring and retrieval of viable CTCs or other target cells for further analysis. The ease of use and portability of the device presents an attractive replacement to current methodologies. A high throughput ensures fast recovery time of diagnosis results which can aid in timely drug administering to better the chances of survival.

A device in accordance with embodiments is fabricated in accordance with the biorheology of cells and depends on the deformability and dimensions of the cells. Studies of various different types of cells' biorheology may be used to determine the dimensions of the device. In such studies, fresh cell samples may be used to ensure consistent biorheological properties of the cells. In accordance with embodiments, there is provided a micro-fluidic device to isolate target cells such as viable cancer cells of breast and colon origins from whole blood using solely the biorheological property differences of cancer cells and blood constituents. No functional modifications of the micro-fluidic device are required as isolation is solely dependent on the biorheological property differences of the target cells such as cancer cells and blood constituents. Past studies have revealed that the shear modulus, stiffness, size and/or deformability of diseased cells (L. Weiss, Adv. Cancer Res. 54, (1990); L. Weiss, D. S. Dimitrov, J Theor. Biol. 121,307 (1986)) is distinctively different from blood constituents (H. Mohamed, L. D. McCurdy, D. H. Szarowski, S. Duva, J. N. Turner, M. Caggana, IEEE Trans. Nanobioscience 3, 251 (2004); J. P. Shelby, J. White, K. Ganesan, P. K. Rathod, and D. T. Chiu, Proc. Natl. Acad. Sci. U.S.A. 100, 2003). The adopted approach draws upon this dissimilarity to achieve high purity in isolating cancer cells in blood. A feasibility study has also been successfully conducted to separate samples based on biorheological differences (S. J. Tan., L. Yobas, G. Y. H. Lee, C. N. Ong, C. T. Lim, International Conference on Biocomputation, Bioinformatics, and Biomedical Technologies, 2008. BIOTECHNO '08 (2008)). The isolation process is achieved in a single step, preserving the integrity and viability of these cells. Retrieval of the isolated cells is also straightforward by controlling and manipulating the flow conditions in a device according to embodiments.

The setup of the device consists of two main components to process samples such as blood. A pressure control component maintains the desired pressure setting through software control on a computer which in turn drives the blood sample into the device or draws the blood sample through the device. The device works on the physical characteristics of cells such as cancer cells from blood cells which are impeded in a cell isolation region. Pre-filters are added to account for coagulations that might be present in blood and a change of pressure settings will allow the recovery of target cells into the collection point. Direct immunofluorescence staining in the device is also possible for enumeration and purity characterization.

The micro-fluidic device may be used to capture and isolate target cells such as circulating tumor cells from peripheral blood of cancer patients for diagnostic and prognostic purposes. The micro-fluidic device is attractive for applications in oncology research, particularly prognostication and prediction of drug response. Embodiments overcome the technical challenges posed by the low cancer cell count in blood combined with large sample volumes. By utilizing the biomechanical property differences of cancer cells from blood, embodiments achieve an effective isolation of cancer cells.

### OVERVIEW

As will be explained in more detail below, in order to trap sufficient numbers of cells, biomechanical means to isolate the target cells need to be provided. This presents a problem in the practical application of biomechanical isolation, since in order to trap sufficient numbers of target cells, large volumes of blood need to be processed. To be able to process large volumes of blood, it is desirable to increase the size of the micro-fluidic device, but this presents practical difficulties in controlling the flow through parts of the device and, in particular, maintaining a required pressure differential across any structures which are used to mechanically isolate the target cells. Hence, a number of arrangements are provided which facilitate the desired flow and pressure differentials within parts of the micro-fluidic device to enable the device to be scaled up to process larger volumes of a sample, such as blood.

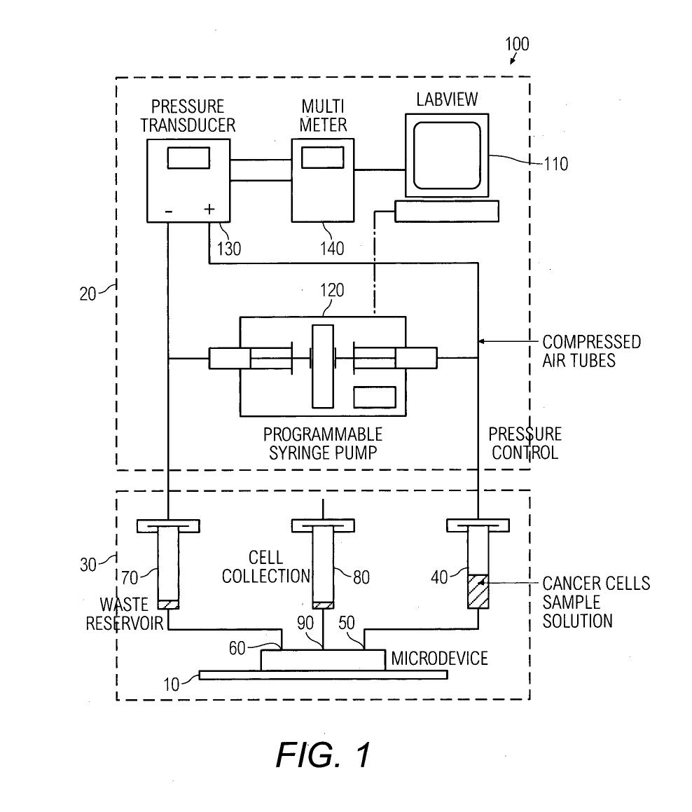

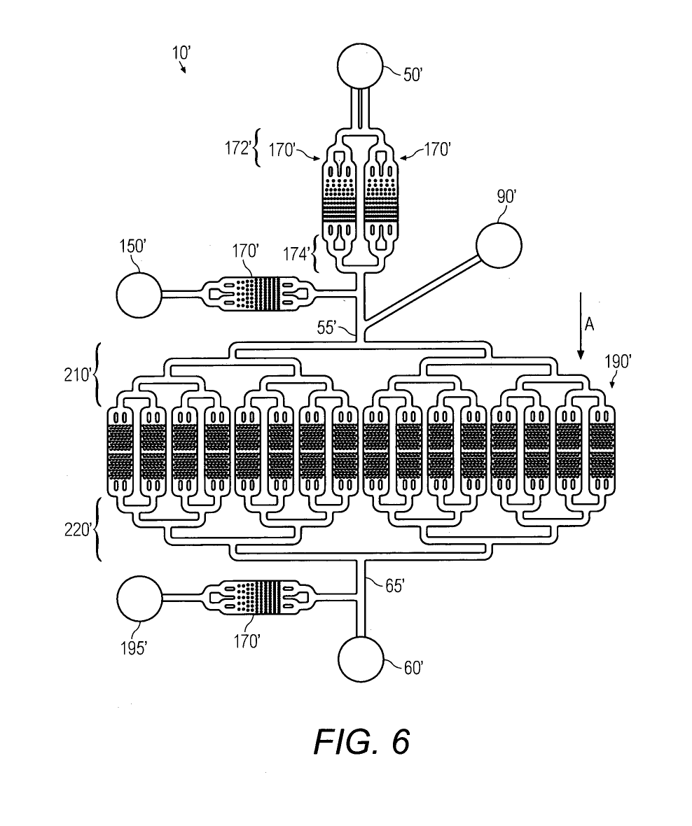

FIG. 1 illustrates a diagnostic system, generally 100, incorporating a micro-fluidic device 10 according to one embodiment. It will be appreciated that the diagnostic system 100 may also be utilized with an alternative micro-fluidic device 10′ illustrated in FIG. 6 below. The diagnostic system 100 comprises a control system 20 coupled with a micro-fluidic device assembly 30 incorporating the micro-fluidic device 10. The micro-fluidic device assembly comprises the micro-fluidic device 10 coupled with a sample syringe 40 containing the sample to be analysed. The sample syringe 40 supplies, under the control of the control system 20, the sample contained therein to a sample inlet 50 of the micro-fluidic device 10. Also coupled with the micro-fluidic device 10 via a waste outlet 60 is a waste syringe 70. The waste syringe 70 is also controlled by the control system 20 and receives fluids emitted from the microfluidic device 10. Also coupled with the micro-fluidic device 10 is a cell collection syringe 80 which receives target cells trapped by the micro-fluidic device 10 provided via a target cell outlet 90. Also coupled with the micro-fluidic device 10 is one or more reagent syringes (not shown) which provide reagents into the micro-fluidic device 10 in order to interact with fluid and/or target cells within the micro-fluidic device to assist subsequent analysis. Also coupled with the micro-fluidic device 10 is one or more blockage syringes (not shown) in which large waste material collected within the micro-fluidic device 10 is collected. The micro-fluidic device assembly 30 consists of a custom made holder which is mountable onto a microscope, sealed tubes and the micro-fluidic device 10.

The control system 20 maintains a desired pressure setting through software control via the computer 110 which drives the programmable syringe pump 120 to apply a pressure differential to the syringes to control fluid flow through the micro-fluidic device 10. In particular, the programmable syringe pump 120 applies a pressure differential to the sample 30 syringe 40 and the waste syringe 70 to control flow from the inlet 50 to the waste outlet 60. The programmable syringe pump 120 may also control the application of reagents into the micro-fluidic device 10 and control the syringes to facilitate the collection of target cells from the micro-fluidic device 10 into the cell collection syringe 80 and facilitate collection of large waste material from the micro-fluidic device 10 into the blockage syringe.

To facilitate control of the syringes, a pressure transducer 130 is coupled with the syringes which, via a multi meter 140 acting as an interfacing device, provides pressure values to the computer 110. Such an arrangement allows a semi-automated process for the sample analysis. The efficiency of the isolation of target cells within the sample is likely to be dependent on the pressure settings used and this arrangement enables such pressure settings to be achieved. Pressure control is favoured over continuous injection, as it prevents a fluid from entering the device should there be a clog due to large debris. This allows for remedial action to be taken so that a potentially precious sample is not wasted. The pressure settings may be monitored on a regular basis and the programmable syringe pump 120 controlled using software (coded in Lab VIEW) to make adjustments, for example every 100 milliseconds. A differential pressure is created in the tubes connected to the micro-fluidic device 10 by compressing or expanding the syringes attached to the syringe pump 120.

In overview, the operation of the micro-fluidic system 100 is as follows. First, the sample to be analysed is placed into the sample syringe 40 which is then coupled with the sample inlet 50. The programmable syringe pump applies a differential pressure to force the sample into the sample inlet 50. The sample passes through the micro-fluidic device 10 which traps target cells therein, as will be explained in more detail below. Any untrapped target cells and other components of the sample emerge from the waste outlet 60 and are received in the waste syringe 70.

Once the required amount of sample has been provided from the sample syringe 40, the sample syringe 40 may be held static whilst one or more reagent syringes are activated to deliver one of more reagents into the micro-fluidic device 10. Surplus fluid is continued to be collected within the waste syringe 70 which may be expanded to assist in maintaining a required pressure differential and flow direction. Once any required reagents have been introduced, the target cells trapped within the micro-fluidic device 10 may then be directly analysed in situ. Additionally or alternatively, the trapped target cells may be recovered from the micro-fluidic device 10. Typically, this is achieved by changing the direction of flow through the micro-fluidic device 10 towards the target cell outlet 90 for storage in the cell collection syringe 80. This may be achieved by maintaining the sample syringe 40 in a static configuration whilst expanding the cell collection syringe 80 and either contracting the waste syringe 70 or contracting a syringe containing a benign fluid coupled to a point at or near the waste outlet 60. As will be explained in more detail below, the reverse directed of flow caused by a reverse pressure differential enables the trapped target cells to disengage from structures within the micro-fluidic device 10 and travel to the cell collection syringe 80.

### MICRO-FLUIDIC DEVICE—1^{sT} EMBODIMENT

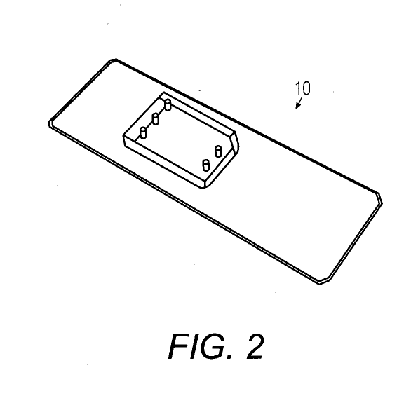

FIG. 2 shows a laboratory prototype of the fabricated micro-fluidic device 10. Fabrication of the device is achieved with soft lithography using a biocompatible polymer (polydimethylsiloxane) bonded onto a glass slide.

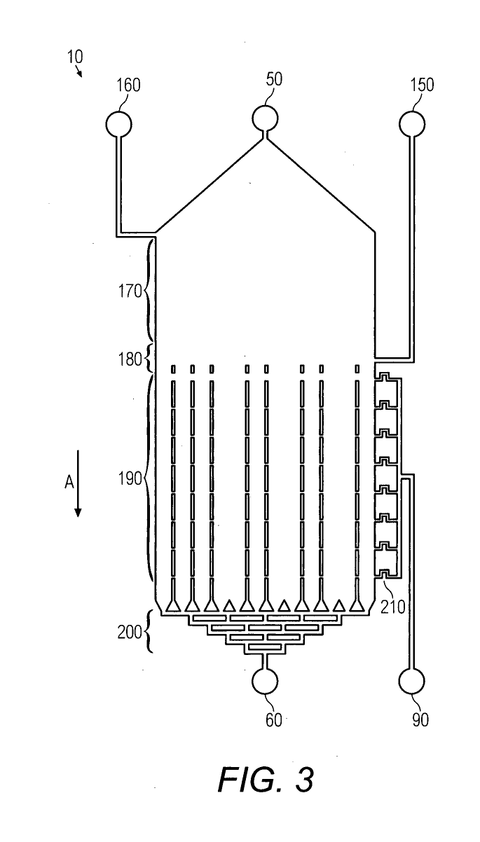

FIG. 3 illustrates an arrangement of the micro-fluidic device 10 in more detail. As can be seen, the micro-fluidic device 10 has a sample inlet 50, a waste outlet 60 and a target cell outlet 90. In addition, the device has the reagent inlet 150 and a debris removal outlet 160. The debris removal outlet 160 is typically coupled with a debris syringe (not shown) to enable large items of debris which are unable to pass through the micro-fluidic device 10 to be removed, as will be explained in more detail below. In addition, trapped emboli, which may also contain CTC may be stained and retrieved via the debris removal outlet 160.

The micro-fluidic device comprises a first pre-filter array 170, which performs initial pre-filtering of the sample to capture large debris such as blood clots or other large bodies, a secondary pre-filter 180, a plurality of arrays of cell isolation structures 190, which capture target cells, and a pressure gradient generator 200. In addition, flow restrictors 210 are provided between the array of cell isolation wells and the target cell outlet 90.

The configuration of the pre-filter arrays 170, 180, together with the arrangement of the gradient generator 200 and the flow restrictors 210, enable a large-scale device having many parallel arrays of cell isolation wells to be provided since a uniform flow and pressure differential across each of those arrays of cell isolation wells can be achieved.

### Device Operation

In overview, the operation of the micro-fluidic device 10 is as follows. Initially, a sample is introduced through the sample inlet 50 and a pressure differential is generated between sample inlet 50 and waste port 60 to cause a flow through the device generally in direction A. This causes the sample being tested to pass through the primary pre-filters 170 and the secondary pre-filters 180. The pre-filters pre-filter the sample, thereby removing the need for the sample to be pre-processed prior to being provided to the micro-fluidic device 10. Any large debris within the sample is likely to be trapped within the pre-filter arrays. Should any particularly large debris be encountered, then a pressure differential may be created to cause a flow out of the debris removal outlet 160, for example by contracting syringes coupled with the sample inlet 50, the reagent port 150 or the waste outlet 60 and by expanding a syringe coupled with the debris removal outlet 160 to dislodge any large bodies which may be blocking flow to remove these from the micro-fluidic device 10. It will be appreciated that such a blockage will be detected by the pressure transducer 130.

The pre-filtered sample then passes into the plurality of arrays of cell isolation wells 190. The cell isolation wells utilise the characteristic that target cells may have different mechanical properties to other cells within the sample. For example, cancer cells may be stiffer and generally larger than most blood cells, which will impede their flow as they pass through the cell isolation structures. The target cells will then be retained within the cell isolation structures whilst other constituents flow past. By maintaining a pressure differential which causes the flow generally in direction A, the target cells remain retained within the cell isolation wells.

Once the sample has been passed through the micro-fluidic device 10 and the sample syringe 40 is typically empty, reagents may then be introduced via the reagent inlet 150. Accordingly, the micro-fluidic device 10 enables in situ immunofluorescence staining of the target cells to quantify and enumerate them or to direct them to the target cell outlet 90 for retrieval. The benefit of on-chip staining simplifies operations by avoiding any intermediate preparatory steps and minimises cell losses due to sample transfer to maximise yield. Furthermore, the reagent use is minimal, as the volume inside the micro-fluidic device 10 is small. For immunofluorescence staining, the sample inlet 50 is closed and the reagent inlet 150 opened. Following standard immunofluorescence protocols, on-chip staining can be completed by flowing in each reagent sequentially. A readout can be achieved manually or using commercial image processing software to identify cells that carry the tumour markers using a normal fluorescence microscope or any compatible array scanner.

Once all on-chip procedures have been completed, the target cells may be removed from the device for external processing or analysis by creating a pressure differential which causes a flow within the arrays of cell isolation wells generally against direction A and towards target cell outlet 90. This may be achieved by expanding the syringe coupled with the target cell outlet 90 and contracting a syringe coupled with waste outlet 60. This dislodges any target cells trapped within the isolation wells and they flow to the target cell outlet 90.

First Pre-Filter Array

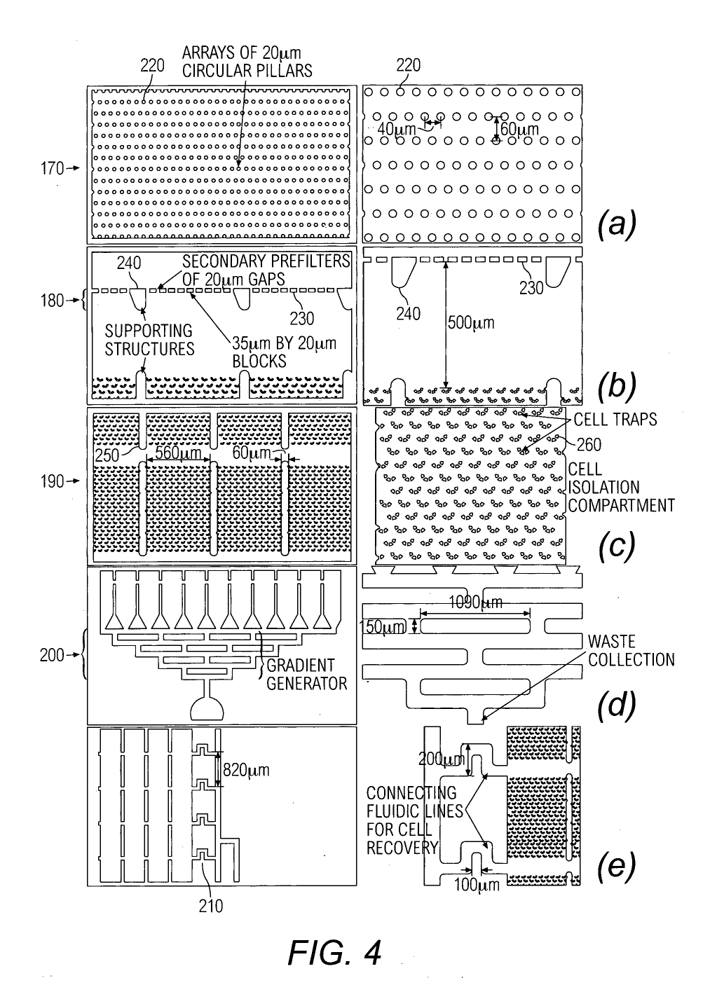

FIG. 4A illustrates the configuration of the first pre-filter 170 array in more detail. Due to considerable clogging in cancer patient blood and also due to the presence of microemboli (tumours fragments that have detached), pre-filtering can be of significant importance. The first pre-filter array 170 comprises an array of structures 220. These structures are formed into an array of rows and columns. Each row is offset from its adjacent column. In this example, the first pre-filter array 170 comprises multiple 20 μm circular structures with gaps of 20 μm between structures in each row. Each adjacent row has a 40 μm gap therebetween. Each adjacent row is offset by another 20 μm to ensure that clumps of large material are held in place. The gaps are sufficiently larger than cells to allow them to pass through the pre-filter array 170 with ease. The distance between each row is set at 60 μm to allow the sample to flow smoothly. Clumps are likely to occur with samples which have been subject to prolonged storage or samples that are improperly stored. The first pre-filter array 170 should effectively remove such clumps before the blood reaches the isolation arrays 190 so that they do not hinder the sample processing. Although this embodiment has a uniform spacing of structures 220, it will be appreciated that the spacing could be arranged to reduce in the direction of flow (i.e. the structures 220 could be cascading, having smaller and smaller gaps therebetween). Likewise, a series of differing structures 220 of different diameters and flow gaps may be provided; these may be correspondingly smaller (as in water filter systems) to trap the microemboli.

### Secondary Pre-Filter

FIG. 4B illustrates the configuration of the secondary pre-filter 180 in more detail. The secondary pre-filter 180 is provided to help to prevent any large debris that may have passed through the first pre-filter 170 from entering the array of cell isolation wells 190. The secondary pre-filter 180 is spaced away from the primary pre-filter array 170 to normalise flow and to equalise any local variation in the pressure caused as a result of any blockages in the first pre-filter array 170. Similarly, the secondary pre-filter array 180 is spaced apart from the arrays of cell isolation wells 190 to provide for such flow and pressure normalisation.

The secondary pre-filter array 180 comprises a row of structures. In this example, there is provided a row of rectangular blocks 230 having dimensions of 35 μm by 20 μm and spaced to have 20 μm gaps therebetween. The blocks 230 are separated by supporting structures 240 which assist in the mechanical integrity of the micro-fluidic device 10 during fabrication. As can be seen, the arrangement of the supporting structures aligns with similar supporting structures which compartmentalise the isolation region into the plurality of arrays of isolation wells 190. This helps to align the flow from the secondary pre-filter 180 to the arrays of isolation wells 190. Although this embodiment has a single row of blocks 230, it will be appreciated that more than one row could be provided either with uniform spacing and/or the spacing could be arranged to reduce in the direction of flow (i.e. the blocks 230 could be cascading, having smaller and smaller gaps therebetween).

Arrays of Cell Isolation Wells

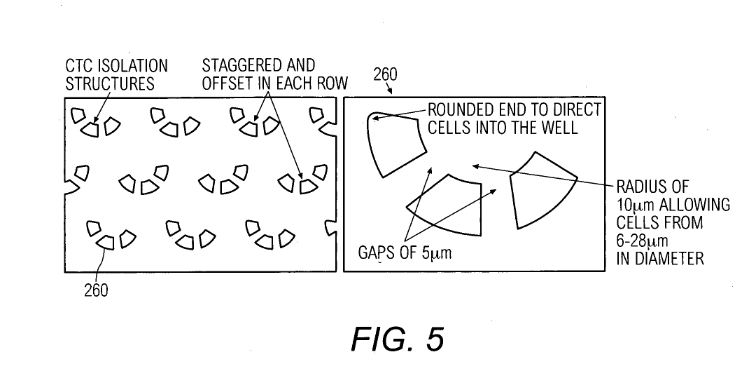

FIG. 4C illustrates an arrangement of the plurality of arrays of cell isolation wells or structures 190 in more detail. In this embodiment, there is provided 8 rows by 12 columns of arrays of cell isolation wells. Each array is compartmentalised by a supporting structure 250. Each array is approximately 560 μm by 850 μm. Each array comprises a number of rows arid columns of isolation structures 260 illustrated in more detail in FIG. 5. The compartmentalisation of the arrays of isolation wells facilitates counting, recovery of target cells and provides uniform flow characteristics. The compartmentalised arrays also facilitate the fabrication of the micro-fluidic device by providing large supporting structures to prevent the device from collapsing. In addition, using compartmentalised arrays facilitates further expansion for future scaling up of the device.

Each compartmentalised array is approximately 560 μm wide and 850 μm in length and holds 200 cell isolation structures 260. Including more isolation structures 260 within each array will affect the recovery process and thus is not favoured. The cell isolation structures 260 in each row are staggered to increase the hydrodynamic efficiency. The dimension and placement of the isolation structures 260 are optimized for fabrication ease and isolation efficiency. Target cells, such as CTCs, are generally less deformable and larger than most blood cells and so will be passively retained by the isolation wells 260 and the multiple rows of isolation wells 260 increases the overall cell retention efficiency. The hydrodynamic profile of the structure also prevents build up of cells in any particular region to facilitate the smooth processing of the samples.

### Isolation Wells

As can be seen in FIG. 5, the placement of the cell isolation wells 260 is staggered to improve the isolation yield and the offset assists in capturing cells that miss an earlier row of cell isolation wells 260. The gaps of around 5 μm and preferably 6 μm to 9 μm within each isolation structure allow constituents to leave the cell isolation wells 260 whilst retaining the target cells in place. In order to trap fetal cells, these gaps may be reduced to 2 or 3 μm. The radius of each cell isolation well 260 is approximately 10 μm to enable entrapment of cells of approximately 6 to 28 μm. The parts of the cell isolation wells 260 facing the direction of flow A are rounded to ensure a smoother transition of the cells into the cell isolation wells 260. Hence, it can be seen that the selection mechanism is based on target cells being stiffer and generally larger than most blood cells which will impede them in the flow as they pass through the cell isolation wells 260. Due to the gaps in between structures, blood constituents are able to deform through which will prevent clogging in the device when processing large volume of blood. Occupied cell isolation wells 260 tend to hold only single or duplet cells due to the design which directs incoming cells downwards when cell isolation wells 260 are filled and this will facilitate counting.

### Gradient Generator

FIG. 4D illustrates in more detail the configuration of the gradient generator 200. The gradient generator 200 is positioned between the plurality of arrays of cell isolation wells 190 and the waste outlet 60. The gradient generator 200 consists of a coupling structure which reduces the number of conduits from a maximum amount at a position closest to the plurality of arrays of cell isolation wells 190 to a single conduit coupled with the waste outlet 60. This tree structure reduces the number of conduits by at least one at each level. This arrangement provides a serial gradient generator that aides the maintenance of uniform conditions in each column of the array of cell isolation wells 190. In particular, the gradient generator 200 provides for a generally constant pressure presented to each column of the array of cell isolation wells irrespective of its relative location with respect to the waste outlet 60. As can be seen, the generator is defined by a plurality of a reducing number of supporting structures dimensioned to generally 190 μm by 150 μm defining channels which are generally 150 μm wide.

### Flow Restrictors

FIG. 4E illustrates in more detail the arrangement of flow restrictors 210 between the plurality of arrays of cell isolation wells 190 and the target cell outlet 90. To facilitate recovery of target cells from within the plurality of arrays of cell isolation wells 190, a flow restrictor 210 is coupled with each row of arrays of cell isolation wells. Each flow restrictor comprises a looped or kinked conduit which changes the direction of flow to increase the fluid resistance and provide a slight back-pressure during, for example, sample processing and reagent application to minimise the presence of any contaminants present in this region during the retrieval of the target cells. Providing multiple couplings with the cell isolation region helps to facilitate the recovery of target cells.

### MICRO-FLUIDIC DEVICE—2ND EMBODIMENT

The device is similar to the device described above but has a reconfiguration of parts, changes in the primary isolation structures and a branching network. In overview, the operation of the micro-fluidic device 10′ is as follows. Initially, a sample is introduced through the sample inlet 50′ and a pressure differential is generated between the sample inlet 50′ and the waste port 60′ to cause a flow through the micro-fluidic device 10′ generally in direction A. This causes the sample being tested to pass through the primary filters 170′, thereby removing the need for the sample to be pre-processed prior to being provided to the micro-fluidic device 10′.

The filtered sample then passes into the plurality of arrays of cell isolation wells 190′. The cell isolation wells utilise the characteristic that target cells may have different mechanical properties to other cells within the sample, in the manner described above. The target cells will then be retained within the cell isolation wells whilst other constituents within the sample flow past. By maintaining a pressure differential which causes the flow generally in the direction A, the target cells remain retained within the cell isolation wells.

Once the sample has been passed through the micro-fluidic device 10′ and the sample syringe is typically empty, reagents may then be introduced via the reagent inlet 150′. Accordingly, the micro-fluidic device 10′ enables in-situ immunofluorescence staining of the target cells to quantify and enumerate them in a similar manner to that described above prior to directing the stained cells to the target cell outlet 90′ for retrieval.

### Primary Filters

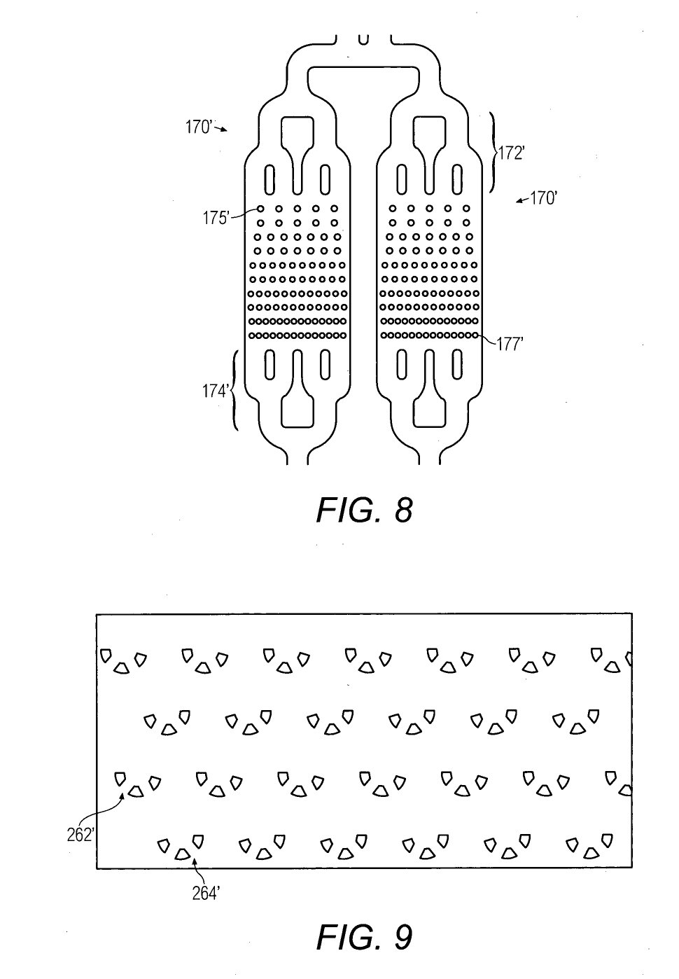

FIG. 8 illustrates the arrangement of the primary filters 170′. In this example, a dual path is provided from the inlet 50′ to a parallel pair of primary filters 170′. Each primary filter 170′ has a branching tree structure 172′ which distributes the sample within the primary filter 170′. In this example, the branching tree structure 172′ takes a single conduit and splits into a dual conduit at each level within the tree. This helps to distribute the sample with uniform flow across the width of the primary filter 170′.

The sample then encounters a series of rows of cylindrical pillars 175′, 177′ which retain debris and cell clumps. The gap between the pillars 175′, 177′ decreases progressively from row to row in the direction A from a starting gap of around 100 μm down to a gap of around 30 μm. Also, the diameter of the cylindrical pillars 175′, 177′ may reduce from row to row in the direction A.

The filtered sample is then received by a branching network 174′ of conduits which combine to a single conduit 55′ which then couples with the parallel arrays of cell isolation structures 190′. It will be appreciated that the branching network may instead reduce to a plurality of conduits.

The primary filters 170′ are placed away from the parallel arrays of cell isolation structures 190′ in order to prevent any local flow disturbances caused by debris or cell clumps trapped by the filter 170′.

Although a single primary filter 170′ could be provided, in this example, a parallel set of primary filters 170′ is provided in order that should one primary filter 170′ become clogged during operation, the sample can still continue to flow through another primary filter 170′.

As can be seen in FIG. 6, additional primary filters 170′ are included to prevent debris or other foreign material from entering the micro-fluidic device 10′ from the reagent port 150′ of from the buffer port 195′. Hence, a primary filter 170′ is present in the inlet port 50′, the reagent port 150′ and the buffer port 195′. These avoid debris entering the chambers. Debris can include dust which makes flow non-uniform in cell isolation chambers.

### Arrays of Cell Isolation Wells

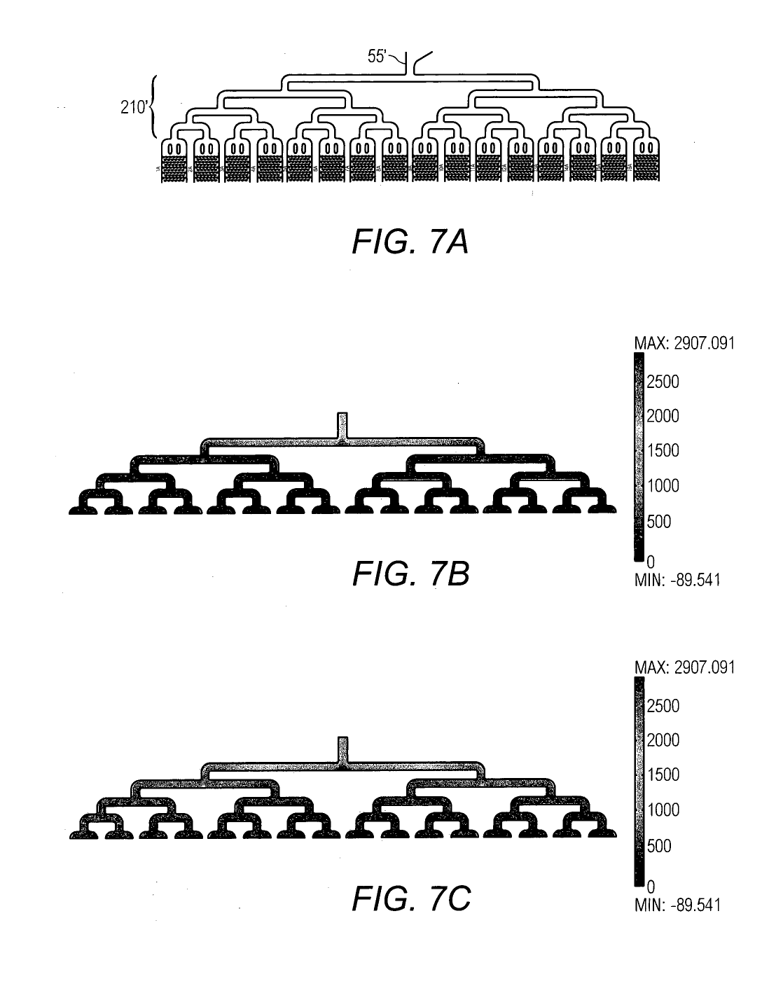

FIG. 7 A shows a portion of the plurality of arrays of cell isolation wells 190′ in more detail. FIG. 7B shows the pressure within a branching network 210′ when fluid is flowing in direction A. In this embodiment, there is provided 16 parallel arrays of cell isolation wells 190′. However, it will be appreciated that greater or few parallel arrays could be provided. Such an arrangement of parallel arrays of cell isolation wells 190′ achieves a processing speed of around 2-6 mL per hour.

The filtered sample is received at the conduit 55′ and is distributed through the branching network 210′ to achieve uniform flow through each array of cell isolation wells. As can be seen, the branching network 210′ forms a tree structure where each conduit splits to two conduits (although each conduit could split to more than two conduits) at each level in the tree. Accordingly, in this example, to expand the single conduit 55′ to feed all 16 parallel arrays of cell isolation wells requires four levels of the tree. This branching network of inlet channels affects throughput and helps to distribute flow evenly. The flow profile is generally non-uniform as it enters the device and this is ameliorated by the branching network. The branching network increases the number of conduits at each level from 1−>2−>4−>8−>16. It is possible to use 16 conduits or more but generally a minimum 8 conduits are required to give an even flow. 16 conduits give at least 2.5 ml sample per hour.

As can be seen in more detail in FIG. 6, a reverse arrangement branching network 220′ is provided which combines the waste material received from the array of cell isolation wells 190′. This waste is then received at the conduit 65′ and flows under pressure into the waste outlet 60′.

### Cell Traps

As can be seen in more detail in FIG. 9, the arrays of cell isolation wells 190′ consist of crescent-shaped cell traps 262′, 264′ to isolate and capture cells. The cell traps 262′, 264′ define two or more gaps in the crescent shape with a size ranging from around 6 μm to around 9 μm. The cell traps 262′, 264′ are tilted left and right handedly to increase capture efficiency. The distance between individual cell traps 262′, 264′ is optimized to increase flow rate through the micro-fluidic device 10′. The rows of cell traps 262′, 264′ are spaced to increase the probability of cell capture. The distance between successive rows of cell traps 262′, 264′ increases to increase the processing throughput.

### Cell Retrieval

In order to retrieve the captured cells, the direction of flow within the micro-fluidic device 10′ is reversed within the arrays of cell isolation wells 190′. This is achieved by preventing any flow in or out of the inlet 50′, the reagent port 150′ or the waste outlet 60′. Fluid is introduced through the buffer port 195′, which is filtered by a filter array 170′.

The fluid received through the buffer ports 195′ is received within the conduit 65′ and is distributed by the reverse arrangement branching network 220′ and into the parallel arrays of cell isolation structures 190′. This reverses the flow direction (to generate a flow in a direction which is opposite to the direction A) and dislodges any cells trapped within the cell traps 262′, 264′. FIG. 7C shows the pressure within a branching network 210′ when fluid is flowing in a direction which is opposite to direction A. The dislodged cells then travel into the branching network 210′ and are received at the conduit 55′. A flow is then established toward the target outlet 90′ and the cells are retrieved via the target cell outlet 90′.

Using this arrangement it is possible to place the target cell outlet 90′ at the top of the cell isolation region (on the inlet side parallel arrays of cell isolation structures 190′) rather than at the side of the cell isolation region. By placing the target cell outlet 90′ at the top rather than at the side, a uniform flow field can be created inside the parallel arrays of cell isolation structures 190′ to enable efficient cell removal.

Placing the buffer port 195′ at the bottom of the parallel arrays of cell isolation structures 190′ assists in evenly distributing the flow through the parallel arrays of cell isolation structures 190′ during cell retrieval. To assist in the cell removal, the pressure differential is maintained between the buffer for 195′ and the target cell outlet 90′. In order to eliminate cell loss, no filters are included between the parallel arrays of cell isolation structures 190′ and the target cell outlet 90′.

Hence, rather than placing the target cell outlet 90′ at the side of the chip which can result in a non-uniform flow profile where there is only flow on the target cell outlet side (right hand side) of device and no flow on the other side (left hand side) of the device which results in only cells in isolation wells on the target cell outlet side being flushed out and those on the other side of the device remaining trapped, the target cell outlet 90′ is instead placed the top of the device (i.e., on the same side as the inlet ports). The buffer port 195′ is placed at the bottom of device. After trapping, buffer solution is pushed from buffer port 195′ at the bottom to flush cells out to target cell outlet 90′ at top. With this embodiment, flow is evenly distributed across the device, so that there is even dislodgement and more cells are flushed out so that the efficiency of isolation is increased.

It will be appreciated that features of the embodiment shown in FIG. 6 may be combined with features of the embodiment shown in FIG. 2.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cell and molecular biology or related fields are intended to be within the scope of the claims.

What is claimed is:

1. A micro-fluidic device operable to isolate target cells from a biological fluid, said micro-fluidic device comprising:
an inlet operable to receive said biological fluid, said biological fluid comprising target cells and other components;
an waste outlet operable to receive at least said other components of said biological fluid;
a plurality of parallel arrays of cell isolation traps coupling said inlet with said waste outlet, each parallel array of cell isolation traps supporting a flow of said biological fluid from said inlet to said waste outlet in response to a pressure differential thereacross, each cell isolation trap being dimensioned to mechanically trap said target cells therein whilst permitting flow of other components of said biological fluid;
at least one pressure maintenance structure operable to assist in maintaining a predetermined pressure differential across each of said plurality of parallel arrays of cell isolation traps,
a buffer port for reversing the flow through the plurality of parallel arrays of cell isolation traps, and
a target cell recovery outlet for retrieving the trapped target cells during the reversed flow.

2. The micro-fluidic device of claim 1, wherein the target cells are CTCs and the biological fluid is whole blood.

3. The micro-fluidic device of claim 2, wherein the cell isolation trap comprises a crescent-shaped structure operable to retain the target cells.

4. The micro-fluidic device of claim 3, wherein the cell isolation trap further comprises at least one gap in the crescent-shaped structure.

5. The micro-fluidic device of claim 4, wherein the at least one gap is between 6 μm to 9 μm and configured to trap substantially viable CTCs and to pass more deformable cells including white blood cells.

6. The micro-fluidic device of claim 3, wherein the crescent-shaped structures are tilted with a tilt axis which is non-orthogonal with respect to a direction of flow through the plurality of parallel arrays of cell isolation traps.

7. The micro-fluidic device of claim 6, wherein each cell isolation trap within each row of cell isolation traps is tilted with the same tilt axis.

8. The micro-fluidic device of claim 7, wherein each cell isolation trap within adjacent rows of cell isolation traps is tilted with opposing tilt axes.

9. The micro-fluidic device of claim 1, wherein cell isolation traps within each row are spaced apart by gaps and cell isolation traps within adjacent rows are positioned to align with the gaps.

10. The micro-fluidic device of claim 1, wherein a distance between adjacent rows of cell isolation traps increases in a direction of flow from the inlet to the outlet.

11. The micro-fluidic device of claim 1, wherein said at least one pressure maintenance structure comprises:
a primary pre-filter array positioned between said inlet and said plurality of parallel arrays of cell isolation traps, said primary pre-filter array comprising a plurality of rows of primary structures dimensioned to mechanically trap bodies within said biological fluid larger than said target cells.

12. The micro-fluidic device of claim 11, wherein said at least one pressure maintenance structure comprises:
a secondary pre-filter array positioned between said primary pre-filter array and said plurality of parallel arrays of cell isolation traps, said secondary pre-filter array comprising at least one row of secondary structures dimensioned to mechanically trap bodies within said biological fluid larger than said target cells.

13. The micro-fluidic device of claim 1, wherein said at least one pressure maintenance structure comprises:
a flow combiner positioned between said plurality of parallel arrays of cell isolation traps and said waste outlet and, said flow combiner comprising a tree of fluidic channels operable to maintain a uniform pressure presented by said outlet to each of said plurality of parallel arrays of cell isolation traps.

14. The micro-fluidic device of claim 13, wherein a root level of said tree presents one fluidic channel to said waste outlet and a leaf level of said tree presents ‘n’ fluidic channels to said plurality of parallel arrays of cell isolation traps.

15. The micro-fluidic device of claim 14, wherein each leaf level away from said root level and towards said plurality of parallel arrays of cell isolation traps presents an additional fluidic channel.

16. The micro-fluidic device of claim 1 wherein said at least one pressure maintenance structure comprises:
a conduit coupling said plurality of parallel arrays of cell isolation traps with said target cell recovery outlet, said conduit being shaped to restrict flow of fluid therethrough.

17. The micro-fluidic device of claim 16, wherein said conduit is shaped to restrict flow of fluid by causing at least one change in direction of flow.

18. The micro-fluidic device of claim 1, comprising:
a reagent inlet operable to receive a reagent, said reagent inlet comprising a conduit operable to deliver said reagent to between said inlet and said plurality of parallel arrays of cell isolation traps.

19. The micro-fluidic device of claim 1, wherein the cell isolation trap comprises three spaced pillars, each pillar having a substantially high aspect ratio.

20. A micro-fluidic device operable to isolate target cells from a biological fluid, said micro-fluidic device comprising:
an inlet operable to receive said biological fluid, said biological fluid comprising target cells and other components;
an waste outlet operable to receive at least said other components of said biological fluid;
a plurality of parallel arrays of cell isolation traps coupling said inlet with said waste outlet, each parallel array of cell isolation traps supporting a flow of said biological fluid from said inlet to said waste outlet in response to a pressure differential thereacross, each cell isolation trap being dimensioned to mechanically trap said target cells therein whilst permitting flow of other components of said biological fluid;
at least one pressure maintenance structure operable to assist in maintaining a predetermined pressure differential across each of said plurality of parallel arrays of cell isolation traps,
wherein the cell isolation trap comprises a crescent-shaped structure operable to retain the target cells, each crescent-shaped structure is tilted with a tilt axis which is non-orthogonal with respect to a direction of flow through the plurality of parallel arrays of cell isolation traps, and each crescent-shaped structure within adjacent rows of crescent-shaped structures is tilted with opposing tilt axes.
